(19) United States
(12) Patent Application Publication
(10) Pub. No.: US 2008/0171948 A1
(43) Pub. Date: Jul. 17, 2008

(54) SUBDERMAL NEEDLES

(76) Inventor: Sidney M. Broder, Birmingham, MI (US)

Correspondence Address: BUTZEL LONG;IP DOCKETING DEPT, 350 SOUTH MAIN STREET, SUITE 300, ANN ARBOR, MI, 48104, US

(21) Appl. No.: 11/622,355

(22) Filed: Jan. 11, 2007

(51) Int. Cl. A61B 5/0478

(52) U.S. Cl. 600/544

(57) ABSTRACT

Subdermal needle electrode assemblies that include needle electrodes and connectors for releasably coupling electrical leads to the needle electrodes. The needle electrodes and connectors can be single-use, disposable items and the electrical leads can be reusable. The connectors and needle electrodes can be configured so that the connectors can be clipped or snapped onto the electrical leads electrodes or plugged into or onto the electrical leads. In use the needle electrodes can be inserted through the skin of a patient before the electrical leads are connected to the needle electrodes and used to monitor neurological events, including sensory impulses.

### TECHNICAL FIELD

The present invention relates to electrodes that are used to monitor neurological events such as nerve, spinal core and brain function during testing and surgical procedures. In particular, the present invention relates to subdermal needle electrodes for monitoring neurological events including as nerve, spinal core and brain function which subdermal needle electrodes have detachable electrical leads.

### BACKGROUND ART

Needle electrodes are well known for their use in neurologic examinations using monitoring equipment, such as EMG monitoring equipment. In addition, needle electrodes are used to pick up sensory impulses on the scalp of patients that are generated by the brain and spinal cord. Needle electrodes are inserted beneath a patient's skin at locations that are to be stimulated, probed or monitored and then connected to monitoring equipment. As needle electrodes are inserted into a patient's body, both the needle electrode and the operator may be exposed to bodily fluids carrying infectious diseases. Because of the danger associated with exposure to bodily fluids, the Food and Drug Administration (FDA) has stringent requirements regarding packaging, sterilization, reuse, disposal and testing/monitoring performed using needle electrodes.

FDA requirements have resulted in two types of needle electrodes that are currently in use. The first type of needle electrode generally used is a disposable needle electrode assembly that includes a needle electrode, lead wires, and electrical connectors all joined to form a single unit. Such needle electrode assemblies are generally sterilized and prepackaged for one-time use by a physician. The physician opens the sterilized package, inserts the needle electrode into a patients muscle and connects the needle electrode assembly to the appropriate monitoring equipment. After testing/monitoring is complete, the entire needle electrode assembly is disconnected from the monitoring equipment and discarded.

Disposable needle electrode assemblies are expensive and highly wasteful of resources due to their one-time use. Disposable needle electrode assemblies also add to the quantity of medical waste produced, increasing waste disposal costs. Because disposable needle electrode assemblies are prepackaged for a specific use, i.e., specific needle size, length, etc., a hospital must stock large numbers of needle electrode assemblies having different sizes and lengths, adding to inventory overhead and storage costs.

The second type of needle electrode assembly commonly used is a reusable needle electrode assembly designed to be sterilized after each use. Similar to disposable needle electrode assemblies, reusable needle electrode assemblies are generally manufactured as a single piece unit including electrical connectors, leads, and needle electrode. Reusable needle electrode assemblies are not as wasteful of resources; however, they are expensive and time-consuming to use due to the complex procedures necessary to sterilize the needle electrode assemblies prior to each use. As with disposable needle electrode assemblies, reusable needle electrode assemblies are generally manufactured as a single unit having a needle electrode of a specific size and length, thus requiring a hospital to stock a wide variety of needle electrode assemblies.

U.S. Pat. No. 5,482,038 to Ruff discloses a reusable needle electrode assembly that is configured to releasably engage and hold needle electrodes of various configurations. To prevent this needle electrode holder from contacting a patient's skin and thus possibly a patient's bodily fluids during use, the same inventor has developed various needle electrode configurations that prevent the needle electrode holder from contacting a patient's body or fluids. These needle electrode configurations are disclosed in U.S. Pat. No. 5,593,429 to Ruff. Preventing the needle electrode holder from coming into contact with a patients body and bodily fluids eliminates the need to sterilize the holder, decreasing the time and costs associated with using the needle electrode holder.

Subdermal needle electrodes are usually composed of a solid metal (such as tungsten, stainless steel, platinum, etc.). They can be inserted subdermally and provide an immediate biopotential signal. The quality of the recording is reasonable but they are prone to artifact and usually have high impedance. They are usually considered to be acute electrodes (minutes, hours) as their rigid properties limit their application on freely moving subjects.

Various types of subdermal needle electrodes are commercially available such as low profile, straight, right angle, disposable (single use) and hook needles to name a few. In general, subdermal needle electrodes that are commercially available comprise, as a single unit, a needle electrode, lead wires, and electrical connectors that are all permanently joined together.

The present invention provides subdermal needle electrode assemblies that have removable lead wires.

### DISCLOSURE OF THE INVENTION

According to various features, characteristics and embodiments of the present invention which will become apparent as the description thereof proceeds, the present invention provides a medical needle electrode assembly for subdermal use which includes:

a sterilized needle having a distal end for insertion though the skin of a patient and an opposite proximal end;

an electrical lead; and

a connector for releasably coupling the electrical lead to the proximal end of the sterilized needle.

The present invention further provides a method of monitoring neurological events in a patient which involves the steps of:

a) providing a medical needle electrode assembly that includes:
a sterilized needle having a distal end for insertion though the skin of a patient and an opposite proximal end;
an electrical lead; and
a connector for releasably coupling the electrical lead to the proximal end of the sterilized needle;

b) inserting the distal end of the sterilized needle though a patient's skin;

c) connecting the electrical lead to the proximal end of the sterilized needle; and

c) monitoring neurological events from electrical signals obtained by the electrical lead.

The present invention further provides set of medical needle electrode assembles for subdermal use which includes:

a plurality of sterilized needles having distal ends for insertion though the skin of a patient and opposite proximal ends;

a plurality of different colored electrical leads; and

a plurality of connectors for releasably coupling the electrical leads to the proximal ends of the sterilized needles.

### BRIEF DESCRIPTION OF DRAWINGS

The present invention will be described with reference to the attached drawings which are given as non-limiting examples only, in which:

FIG. 1 is a perspective view of a subdermal needle electrode assembly according to one embodiment of the present invention.

FIG. 2 is a top view of a subdermal needle electrode assembly with the electrical lead separated from the subdermal needle.

FIG. 3 is a top view of a subdermal needle electrode assembly with the electrical lead connected to the subdermal needle.

FIG. 4 is a side elevational view of the subdermal needle assembly of FIG. 4.

FIGS. 5 and 6 depict how the subdermal needle is coupled to the electrical lead according to one embodiment of the present invention.

FIG. 7 is a perspective view of a subdermal needle electrode assembly according to another embodiment of the present invention.

### BEST MODE FOR CARRYING OUT THE INVENTION

The present invention is directed to subdermal needle electrodes for monitoring neurological events such as nerve, spinal core and brain function which subdermal needle electrodes have detachable electrical leads. The subdermal needle electrodes of the present invention have distal ends which are configured, e.g. bluntly pointed, to be inserted through the skin of a patient and opposite proximal ends that are configured to be detachably coupled or connected to electrical leads. According to one embodiment the proximal ends of the subdermal needle electrodes are configured so that the electrical leads can be removably clipped thereon. In further and alternative embodiments the proximal ends of the subdermal needle electrodes are configured so that the electrical leads can be removably snapped or plugged onto or into proximal ends of the subdermal needle electrodes. Further, the proximal ends of the subdermal needle electrodes could be configured to be mechanically and removably coupled to ends of the electrical leads using any convenient coupling structure without being limited to clips, snaps, or plug-in structures.

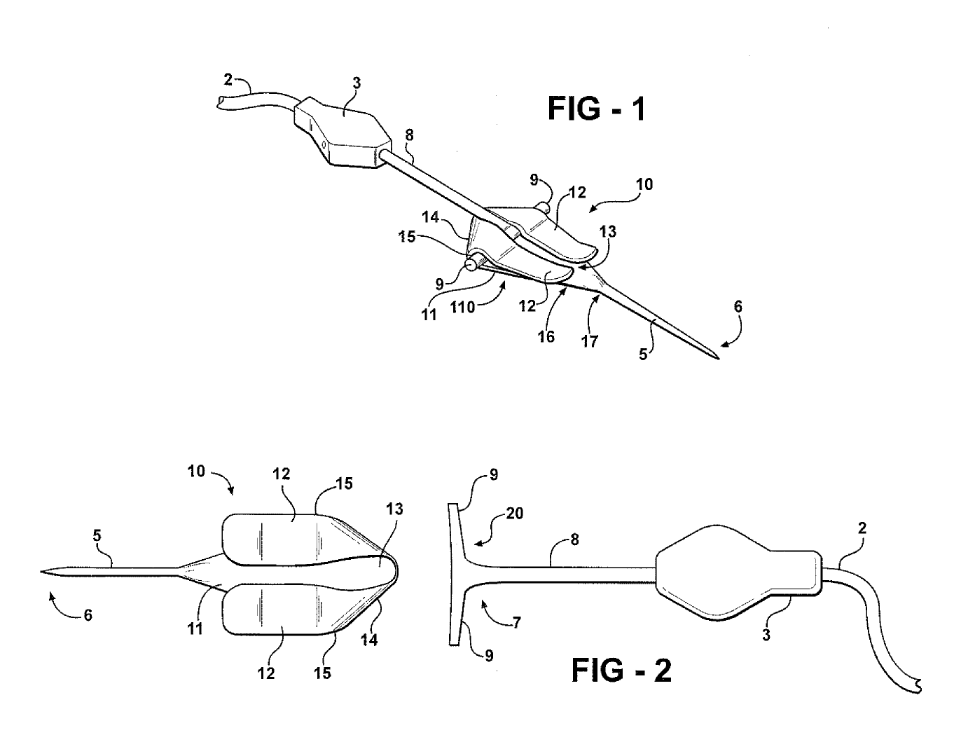

FIG. 1 is a side elevational view of a prior art subdermal needle electrode. The subdermal needle electrode shown in FIG. 1 includes a needle 1 that is coupled to an electrical lead 2 by a hub 3. The hub 3 comprises a permanent mechanical connection between proximal end of the needle and the electrical lead which is covered by a molded plastic sheath.

FIG. 1 is a perspective view of a subdermal needle electrode assembly according to one embodiment of the present invention. The subdermal needle electrode assembly depicted in FIG. 1 includes a needle 5 having a distal end 6 and a proximal end 7. The distal end 6 of the needle 5 is bluntly pointed so that the distal end 6 can be inserted through and beneath the skin of a patient and maintained at a desired position. The proximal end 7 of needle 5 is coupled to the distal end of a connector or clip 10. The proximal end 7 of needle 5 can be attached in any suitable manner to the distal end 16 of the connector or clip 10, or can be formed integrally with the connector or clip 10. As show, the distal end 16 of the connector or clip 10 can have a tab or projection from which the needle 5 can extend. In the embodiment of the invention depicted in FIGS. 1-6, the distal end 20 of the electrical lead 8 has a general “T-shape” with arms 9 that extend outward from the central axis of the electrical lead 8 and lie in a common plane as depicted. The connector or clip 10 has a base 11 and two wings 12 that are folded back over the base 11 so as to define a pocket between the base 11 and wings 12 into which pocket the arms 9 of the T-shaped portion of the distal end 20 of the electrical lead 8 can be received. The wings 12 further define a slot 13 through which the electrical lead 8 can extend. As discussed below, the wings 12 of the clip 10 are configured to engage the arms 9 of the T-shaped portion of the distal end 20 of the electrical lead 8 so as to releasable lock the clip 10 onto the distal end 20 of the electrical lead 8. The T-shaped portion of the distal end 20 of the electrical lead 8 and at least a short length at the distal end thereof is made from a suitable rigid material that has sufficient mechanical strength to prevent the electrical lead from breaking while the remaining length of the electrical lead 8 can be made from conventional gage wire. As depicted, electrical lead 8 is coupled to a length of an electrical lead wire 2 by a hub 3 which comprises a permanent mechanical connection between the proximal end of electrical lead 8 and the electrical lead wire 2 which is covered by a molded plastic sheath.

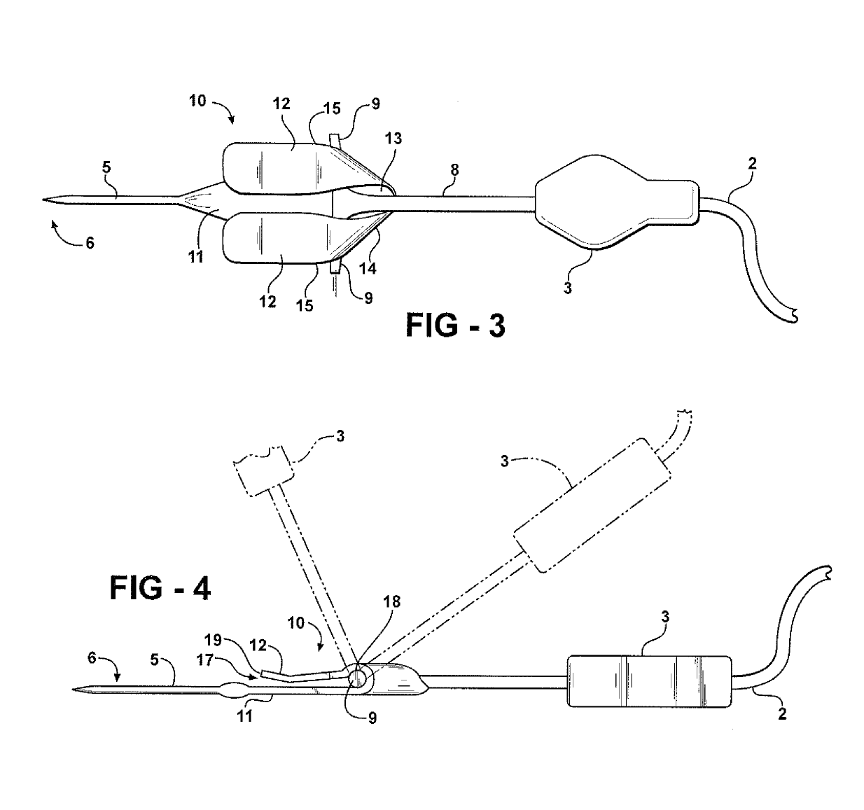

FIG. 2 is a top view of a subdermal needle electrode assembly with the electrical lead separated from the subdermal needle. FIG. 4 is a top view of a subdermal needle electrode assembly with the electrical lead connected to the subdermal needle. As shown in FIGS. 2 and 3, the wings 12 of the clip 10 are folded back at the proximal end 14 of the clip 10 to overlap the base portion 11 of the clip 10. Slot 13 defined between the wings 12 is as wide as or slightly wider than the diameter of the electrical lead 8 so as to allow the electrical lead 8 to pass therethrough as depicted in FIG. 3. As shown in FIG. 3 the arms 9 of the T-shaped portion of the distal end 20 of the electrical lead 8 can slid rearward in clip 10 between the base 11 and wings 12 until the arms 9 of the T-shaped portion of the distal end 20 of the electrical lead 8 abut the edges 15 where the wings 12 of the clip 10 are folded back over the base 11 (and/or engage engaging portions 18 as discussed below). In this position, as further discussed below, the wings 12 of the clip 10 are configured to engage the arms 9 of the T-shaped portion of the distal end 20 of the electrical lead 8 so as to releasable lock the clip 10 onto the distal end 20 of the electrical lead 8.

In order to ensure that good electrical conductivity between the needle 5, clip 10 and T-shaped portion of the electrical lead 8, each can be made of a suitable metal that is conductive, sterilizable and does not pose any health concerns for patient testing or monitoring. In alternative embodiments the clip 10 can be made of materials other than conductive metals and be provided with conductive electrical contacts and leads that provide electrical conductivity between the needle 5 and electrical lead 8 (See FIG. 3). For example, the clip 10 could be made from a plastic material and have electrically conductive surface contacts embedded in or provided on the inner surface or portion of the inner surface of the base portion 11 and/or wings 12 which define the pocket that receives the arms 9 of the T-shaped portion of the distal end 20 of the electrical lead 8. Such an embodiment would allow the clips to be produced by a molding process. It is noted that whereas FIGS. 1, 3 and 6 depict the electrical lead 8 as extending outward from the proximal end 14 of the clip 10, the configuration of the clip 10 also allows the T-shaped portion of the distal end 20 of the electrical lead 8 to be coupled to the clip 10 so that the electrical lead 8 extend outward from the distal end 16 of the clip 10. This ability to change the orientation of the electrical lead 8 with respect to the needle 5 can be used to adjust tension that might be applied through the electrical lead onto the needle after it is inserted into a patient. For example, if the electrical lead 8 will be subject to tension it might be better to orient the electrical lead to extend from the clip 10 in the same direction as the needle 5 so that the tension will tend to secure the placement of the needle 5.

It can be understood from FIGS. 2 and 3 that while the slot 13 through which the electrical lead 8 extends is as long as the wings 12 which define the slot 13 it is only necessary to have a small slot or opening centrally located at the proximal end 14 of the clip 10. Accordingly, according to an alternative embodiment, the clip 10 could include only a small slot or opening centrally located at the proximal end 14 thereof.

FIG. 4 is a side elevational view of the subdermal needle assembly of FIG. 4. By configuring the wings 12 to fold back over the base 11 of the clip 10 the wings 12 can be biased against being deflected away from base 11. As shown in FIG. 4 the gap 17 between the inner surface of the wings 12 and the inner surface of the base 11 of the clip 10 is slightly smaller than the diameter of the arms 9 of the T-shaped portion of the distal end 20 of the electrical lead 8. This dimensional relationship causes the wings 12 to be pushed away from the base 11 as the electrical lead 8 is inserted into clip 10, or more particularly as the arms 9 of the T-shaped portion of the distal end 20 of the electrical lead 8 are slid into the clip 10. The resulting biasing force that urges the wings 12 toward the base 11 is used to releasably lock the clip 10 onto the electrical lead 8. In this regard, the wings 12 are configured to have an engagement portion 18 which is shaped to accommodate the diameter of the arms 9 of the T-shaped portion of the distal end 20 of the electrical lead 8 so as to allow the biasing force in the wings 12 to move toward the base portion 11 as the arms 9 of the T-shaped portion of the distal end 20 of the electrical lead 8 slide into the engagement portions 18. As shown, the distal ends 19 of the wings 12 can be configured to curve away from the base 11 so as to allow the arms 9 of the T-shaped portion of the distal end 20 of the electrical lead 8 to enter gap 17 and further to provide an edge at the proximal ends 19 of the wings 12 that can be lifted away from the base 11 so as to disengage the arms 9 of the T-shaped portion of the distal end 20 of the electrical lead 8 from the engagement portions 18 to remove the clip 10 from the electrical lead 8. FIG. 4 depicts various orientations of the electrical lead 8 with respect to clip 10 which are allowed by pivoting the electrical lead 8 about the an axis that extends through the arms 9 are them are secured in clip 10 as discussed above. As can be appreciated, the electrical lead 8 can be oriented, as desired, to extend outward from either the distal or proximal end of clip 10, or to extend outward in any intermediate position as depicted in FIG. 4.

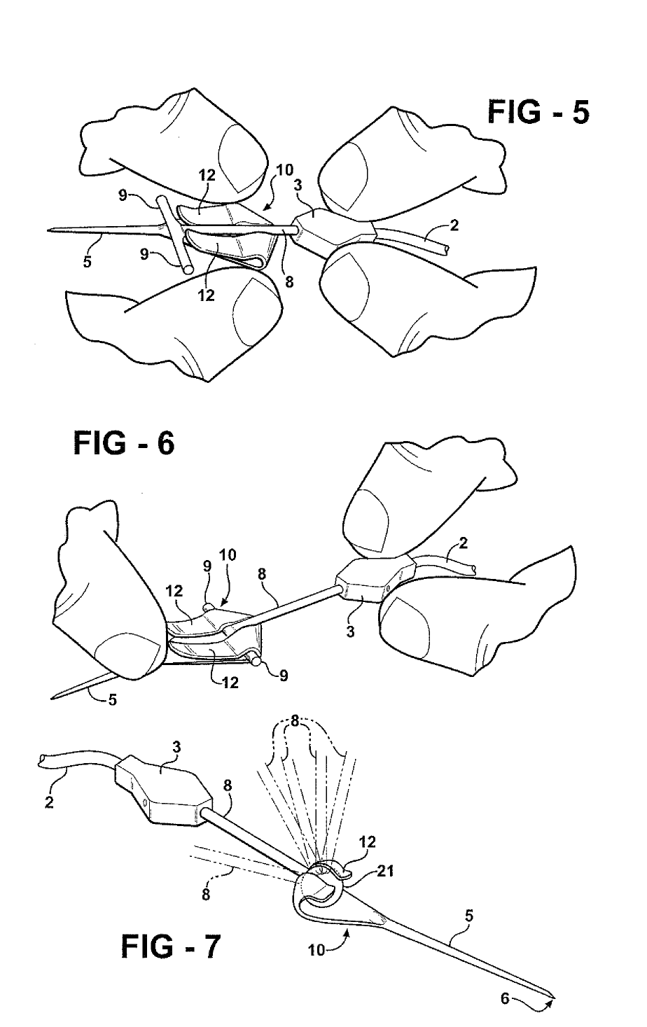

FIGS. 5 and 6 depict how the subdermal needle is coupled to the electrical lead according to one embodiment of the present invention. As shown in FIG. 5 one merely grasps the clip 10 in one hand and the hub 3 at the proximal end of the electrical lead 8 in the opposite hand and aligns the electrical lead 8 so that arms 9 on the distal end 20 of the electrical lead 8 can be pulled beneath distal ends 19 of wings 12. Once aligned, one can pull the electrical lead 8 in the direction of arrows “a” in FIG. 5 and cause the arms 9 on the distal end 20 of the electrical lead 8 to be pulled under wings 12 until the arms 9 abut the edges 15 where the wings 12 of the clip 10 are folded back over the base 11.

FIG. 7 is a perspective view of a subdermal needle electrode assembly according to another embodiment of the present invention. The embodiment of the invention depicted in FIG. 7 includes an electrical lead 8 which has a spherical portion 21 formed at the distal end 20. In addition, the wings 12 are configured to form a spherical cavity into which the spherical portion 21 formed on the distal end 20 of electrical lead 8 can be received and removably secured by sliding the sphere 21 under the distal ends 19 of the wings 12. The use of a spherical connection between the electrical lead 8 and connector or clip 10 allows the electrical lead 8 to be positioned at various angles relative to the radial center of the spherical portion 21 when it is secured in clip 10 as shown in the phantom lines in FIG. 7.

It is to be understood from the above description that the distal end 20 of the electrical lead 8 is not strictly limited to having a T-shape or having a spherical shape, although these shapes offer the ability to adjust the position of the electrical lead 8 with respect to needle 5. In further embodiments, the distal end 20 of the electrical lead 8 and the shape of the clip 10 can be configured in any manner which allows the electrical lead 8 to be removably attached or coupled to the clip 10, including configurations and/or arrangements that allow the electrical lead 8 and connector or clip 10 to be clipped together, snapped together, plugged into one another, or attached or coupled together in any manner which results in being able to detach the electrical lead 8 (for reuse) from the needle 5.

It is noted that while the invention is discussed and illustrated by an embodiment that uses a connector in the form of a clip to couple an electrical lead to the proximal ends of the subdermal needles, in alternative embodiments the proximal ends of the subdermal needles can be configured so that the electrical leads can be removably snapped or plugged onto or into proximal ends of the subdermal needle electrodes. For example, the proximal ends of the needles could be configured to be in the shape of any conventional male or female electrical connector and electrical leads having the corresponding compatible female or male electrical connector structure could be plugged onto or into or snapped or clipped onto the proximal ends of the needles. In general, the proximal ends of the subdermal needle could be configured to be mechanically and removably coupled to ends of the electrical leads using any convenient coupling structure or connector without being limited to clips, snaps, or plug-in structures.

It is further noted that while the present invention is discussed and illustrated with reference to straight subdermal needles, the manner of releasably connecting electrical leads to electrically conductive needles is not limited to any particular type of subdermal needle electrodes or any particular testing or monitoring procedure.

The ability to releasably connect electrical leads to electrically conductive needles according to the present invention provides several advantages over prior art. For example, the subdermal needle electrodes of the present invention can be inserted into a patient before the electrical leads are attached. This avoids problems that be encountered when the electrical leads are permanently attached to subdermal needle electrodes and are accidentally entangled or disturbed causing the position of the subdermal needle electrodes to be adversely affected. Moreover, by only disposing of the needles and reusing the electrical leads, there is a significant savings in costs associated with the present invention.

Although the present invention has been described with reference to particular means, materials and embodiments, from the foregoing description, one skilled in the art can easily ascertain the essential characteristics of the present invention and various changes and modifications can be made to adapt the various uses and characteristics without departing from the spirit and scope of the present invention as described above.

What is claimed is:

1. A medical needle electrode assembly for subdermal use which comprises:
a sterilized needle having a distal end for insertion though the skin of a patient and an opposite proximal end;
an electrical lead; and
a connector for releasably coupling the electrical lead to the proximal end of the sterilized needle.

2. A medical needle electrode assembly for subdermal use according to claim 1, wherein the connector comprises at least one of a clip, snap, or plug-in structure.

3. A medical needle electrode assembly for subdermal use according to claim 2, wherein the distal end of the electrical lead is configured to be releasably coupled to the at least one of the clip, snap or plug-in structure.

4. A medical needle electrode assembly for subdermal use according to claim 3, wherein the distal end of the electrical lead has one of a T-shaped configuration or a spherical shape and the connector comprises a clip complementarily shaped structure.

5. A medical needle electrode assembly for subdermal use according to claim 1, wherein the connector comprises an electrically conductive metal.

6. A medical needle electrode assembly for subdermal use according to claim 1, wherein the connector comprises a molded structure having at least one conductive metal portion.

7. A medical needle electrode assembly for subdermal use according to claim 1, wherein the connector is biased to engage the distal end of the electrical lead.

8. A medical needle electrode assembly for subdermal use according to claim 1, wherein the sterilized needle comprises a subdermal needle.

9. A method of monitoring neurological events in a patient which comprises the steps of:
a) providing a medical needle electrode assembly that includes:
a sterilized needle having a distal end for insertion though the skin of a patient and an opposite proximal end;
an electrical lead; and
a connector for releasably coupling the electrical lead to the proximal end of the sterilized needle;
b) inserting the distal end of the sterilized needle though a patient's skin;
c) connecting the electrical lead to the proximal end of the sterilized needle; and
c) monitoring neurological events from electrical signals obtained by the electrical lead.

10. A method of monitoring neurological events in a patient according to claim 9, wherein the step of connecting the electrical lead to the proximal end of the sterilized needle is conducted after the step of inserting the distal end of the sterilized needle though a patient's skin.

11. A method of monitoring neurological events in a patient according to claim 9, wherein the neurological events or sensory impulses are monitored during a surgical procedure.

12. A method of monitoring neurological events in a patient according to claim 9, wherein the neurological events or sensory impulses are monitored during a testing procedure

13. A method of monitoring neurological events in a patient according to claim 9, wherein the sterilized needle and the connector are discarded after the monitoring is completed and the electrical lead is retained for use in a subsequent neurological monitoring procedure.

14. A method of monitoring neurological events in a patient according to claim 9, wherein the sterilized needle comprises a subdermal needle.

15. A method of monitoring neurological events in a patient according to claim 9, wherein the connector comprises at least one of a clip, snap, or plug-in structure.

16. A method of monitoring neurological events in a patient according to claim 15, wherein the distal end of the lead electrode has one of a T-shaped configuration and a spherical portion and the connector comprises a complementarily shaped clip structure.

17. A method of monitoring neurological events in a patient according to claim 9, wherein the connector is biased to engage the distal end of the electrical lead.

18. A set of medical needle electrode assembles for subdermal use which comprises:
a plurality of sterilized needles having distal ends for insertion though the skin of a patient and opposite proximal ends;
a plurality of different colored electrical leads; and
a plurality of connectors for releasably coupling the electrical leads to the proximal ends of the sterilized needles.

19. A set of medical needle electrode assembles for subdermal use according to claim 18, wherein the plurality of sterilized needles and connectors are packaged separately from the plurality of electrical leads.

20. A set of medical needle electrode assembles for subdermal use according to claim 18, wherein the plurality of sterilized needles and connectors are disposable and the plurality of electrical leads are reusable.
